(19) United States
(12) Patent Application Publication
(10) Pub. No.: US 2009/0093794 A1
(43) Pub. Date: Apr. 9, 2009

(54) BOLUS TUBE ASSEMBLY

(75) Inventors: Daniel Holtz, Chittenango, NY (US); Rafal J. Pawlowski, Utica, NY (US)

Correspondence Address: TYCO HEALTHCARE - EDWARD S. JARMOLOWICZ, 15 HAMPSHIRE STREET, MANSFIELD, MA, 02048, US

(73) Assignee: TYCO HEALTHCARE GROUP LP, Mansfield, MA (US)

(21) Appl. No.: 11/866,867

(22) Filed: Oct. 3, 2007

(51) Int. Cl. A61M 25/16; A61M 25/00; B29C 65/08

(52) U.S. Cl. 604/523; 156/73.1

(57) ABSTRACT

A tube assembly includes a head component that may be weighted. The tube assembly is formed by welding a head tube to a main tube using a die heated by radiofrequency energy. A head lumen in fluid communication with the main tube and seal between the head lumen and a chamber of the head component are formed generally simultaneously when the head tube is welded to the main tube. At another step, weights or filler members are inserted into the chamber. A rounded tip of the tube assembly is formed by forcing a distal end of the head tube into a die that is heated by radiofrequency energy. Outlet openings in fluid communication with the head lumen are formed in the head tube using a punch device. An inlet adaptor of the tube assembly is welded to main tube using a die that is heated by radiofrequency energy.

### FIELD OF THE INVENTION

The present invention generally relates to a tube with a head constructed for insertion into a body of a patient.

### BACKGROUND OF THE INVENTION

Tubes used to deliver oxygen, food and medication can be inserted through a patient's nose or mouth into a patient's body. For example, a nasogastric feeding tube is used to deliver liquid nutrients and medicine into a patient's stomach. The nasogastric feeding tube is inserted through a nostril of the patient, past the throat and into the patient's stomach. This procedure is called nasogastric intubation.

To facilitate the insertion of the tube through the nostril of the patient and into the stomach, nasogastric tubes typically include a head assembly secured to a distal end of an elongate main tube. An inlet adaptor for connecting a source of liquid nutrients and/or medicine is secured to a proximal end of the main tube by solvent bonding. In one particular type of conventional nasogastric feeding tube, the head assembly includes the following four separate components: 1) a head tube having open proximal and distal ends, 2) weights or filler members received in the tube, 3) a connector securing the proximal end of the head tube to the distal end of the main tube, and 4) a button tip closing off the open distal end of the head tube. The connector is secured to the head tube and to the main tube and the button tip is secured to the distal end of the head tube by solvent bonding.

Although solvent bonding provides a satisfactory bond between the respective components of the feeding tube, the chemicals used in solvent bonding are hazardous and the procedure most be performed under a vented hood. Moreover, the components are very small, making the overall process tedious and work intensive because solvent must be properly applied to the small components. Because such care must be taken to ensure proper bonding, it is more likely that the bonding procedure will not always be performed correctly, thereby leading to the possibility of leaks between the components and an overall higher failure rate in manufacturing.

### SUMMARY OF THE INVENTION

In one aspect of the present invention, a method of forming a tube assembly with a bolus head component comprises providing a head tube with an axial passage having an open proximal end and an open distal end. The proximal end margin of the head tube is welded to a distal end margin of a main tube. A proximal portion of the head tube axial passage is collapsed. A head lumen is formed in the collapsed proximal portion of the head tube. The head lumen is in fluid communication with an axial passage of the main tube and is sealed from fluid communication with a distal portion of the axial passage in the head tube. A distal end of the head lumen is sealed to prevent fluid communication between the head lumen and a distal portion of the axial passage of the head tube disposed distally of the seal. At least one filler member is inserted into the distal portion of the axial passage of the head tube through the distal open end of the head tube. The distal open end of the head tube is closed.

In yet another aspect, a bolus tube assembly comprises an elongate main tube having an axial passage extending to an open distal end of the main tube. A head component is welded to the distal end margin of the main tube by high frequency energy at a main-head weld. The head component is formed from a head tube. The head component includes a head lumen in fluid communication with the axial passage of the main tube, and a chamber. The head lumen is formed within the head tube by material of the head tube melted to seal the head lumen from a portion of the tube defining the chamber. A distal end of the head tube is deformed to close off the distal end and seal the chamber between the head lumen and the distal end. At least one filler member is enclosed within the chamber.

Other features will be in part apparent and in part pointed out hereinafter.

### BRIEF DESCRIPTION OF THE DRAWINGS

FIG. 1 is a perspective of one embodiment of a nasogastric feeding tube constructed according to the principles of the present invention;

FIG. 2 is an enlarged, fragmentary side elevation of the nasogastric feeding tube;

FIG. 3 is an enlarged, fragmentary horizontal section of the nasogastric feeding tube showing a head component;

FIG. 4 is an enlarged, fragmentary section of the nasogastric feeding tube showing an inlet adaptor;

FIG. 5A is a flow chart of the feeding tube manufacturing process;

FIG. 5B is a schematic section of a head die and a main-tube mandrel of a head welding device;

FIG. 6 is a section similar to FIG. 5 with a main tube received on the mandrel and a positioning tool being inserted into the head die;

FIG. 7 is a section similar to FIG. 6 with the positioning tool contacting the main tube and forcing the tube into proper position on the main-tube mandrel;

FIG. 8 is a schematic section of a head-tube mandrel and a head tube as the head-tube mandrel is being inserted into the head tube;

FIG. 9 is a schematic section of the head tube and the head-tube mandrel secured to a driver of the head-welding device, the head tube being received in the head die in an initial position;

FIG. 10 is similar to FIG. 9 with the driver moving the head tube axially into the die into contact with the main tube;

FIG. 11 is a section of a partially formed tube assembly being removed from the head die after completion of the head welding process;

FIG. 12 is a schematic section of the partially formed tube assembly secured to a driver and received in a tip die of a tip-welding/punch device;

FIG. 13 is a section similar to FIG. 12 with the driver moving the head tube into the tip die and a tip of the nasogastric tube assembly being formed in the tip die;

FIG. 14 is a section similar to FIG. 12 with the driver withdrawing the partially formed nasogastric tube assembly from the tip die;

FIG. 15 is a section similar to FIG. 14 with a punch of the tip-weld/punch device forming outlet openings of the nasogastric tube assembly;

FIG. 16 is a section similar to FIG. 15 with the punch being withdrawn from the formed outlet openings;

FIG. 17 is a schematic section of the inlet adaptor being inserted into an adaptor die of an inlet adaptor welding device; and

FIG. 18 is a section similar to FIG. 17 with the inlet adaptor welded to the main tube inside the adaptor die.

Corresponding reference characters indicate corresponding parts throughout the drawings.

### DETAILED DESCRIPTION OF THE DRAWINGS

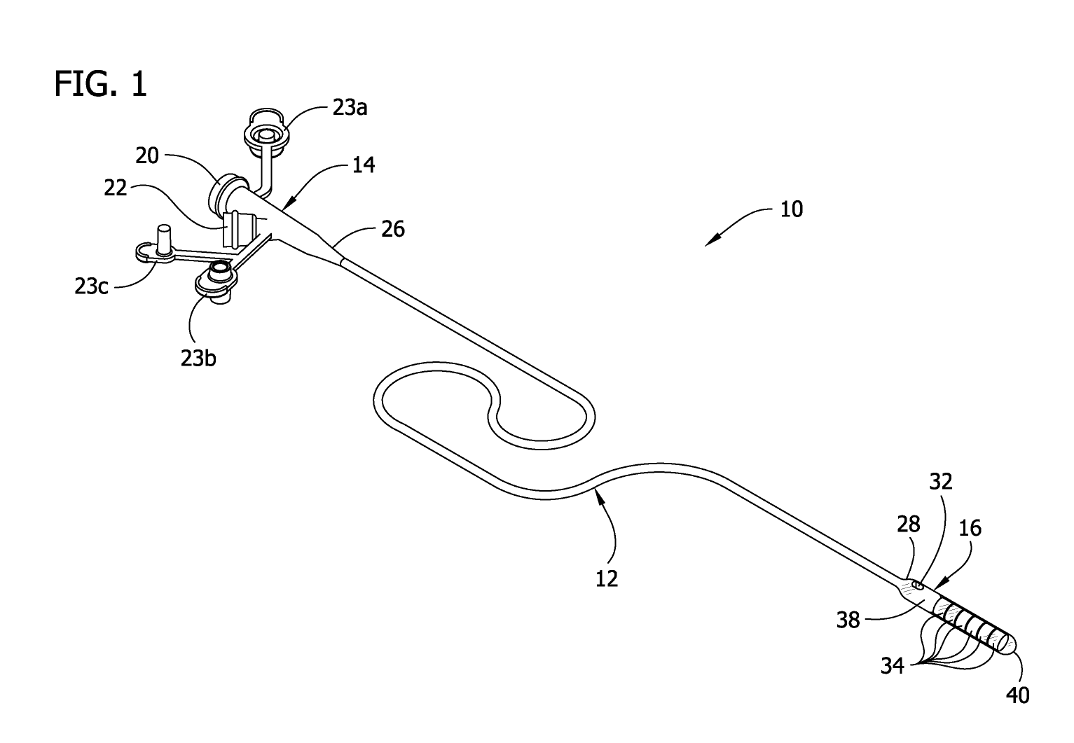

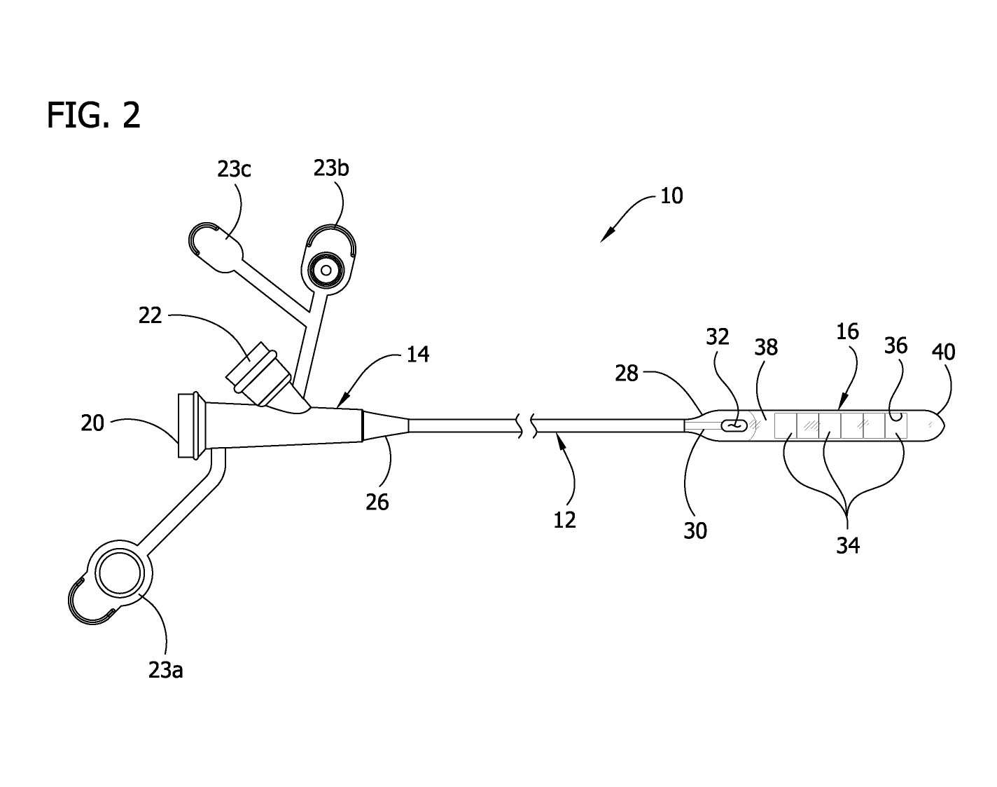

Referring now to the drawings, and in particular to FIGS. 1-3, a nasogastric feeding tube assembly (broadly, a bolus tube assembly) is generally indicated at 10. The feeding tube assembly generally includes a main tube 12, an inlet adaptor 14 secured to a proximal end margin of the main tube, and a bolus head component 16 secured to a distal end margin of the main tube. In the illustrated embodiment, the head component 16 is weighted. The main tube 12, inlet adaptor 14 and head component 16 are indicated generally by their reference numbers. As will be described in detail herein, the inlet adaptor 14 and the weighted head component 16 may be welded to the main tube 12, such as by using high frequency energy (e.g., radiofrequency energy). As used herein, the terms “proximal” and “distal” are used for convenience to describe relative locations of components of the nasogastric feeding tube assembly 10 with respect to a source of liquid nutrients and/or medicine (not shown) that would be connected to the inlet adaptor 14. Thus, for example, the inlet adaptor 14 is the most proximal part of the feeding tube assembly and the head component 16 is the most distal.

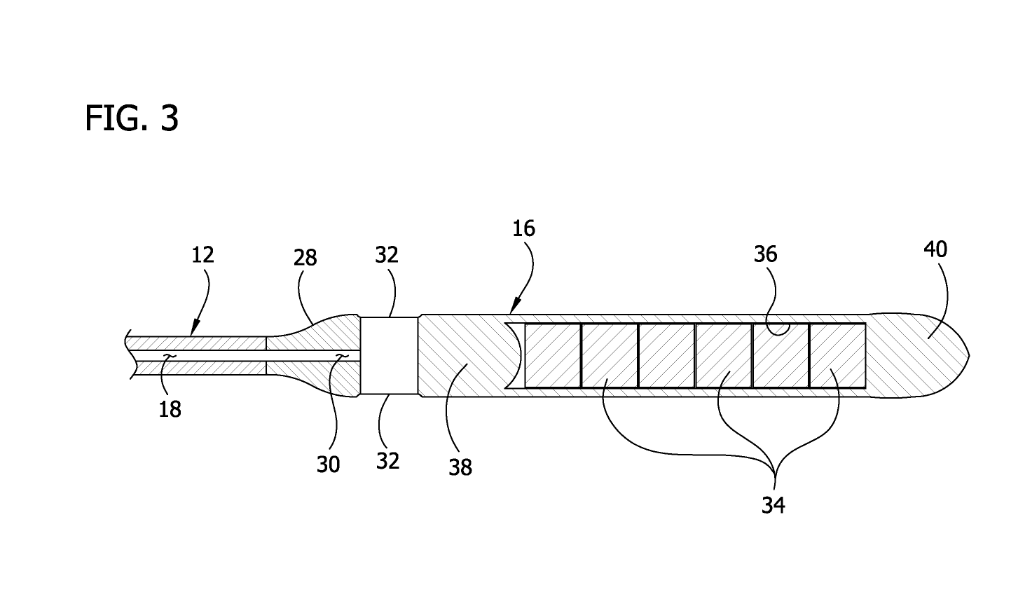

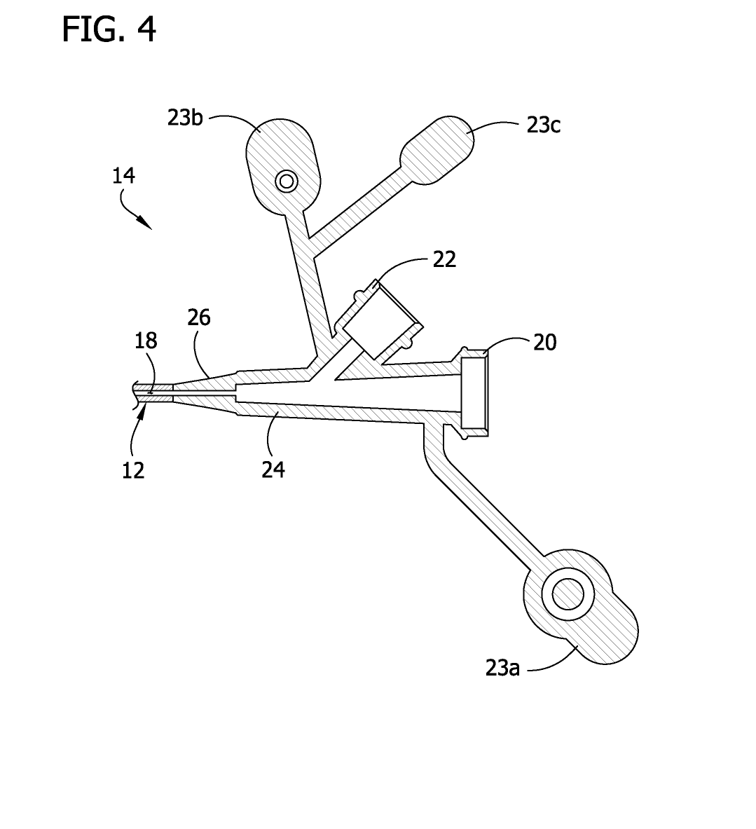

The main tube 12 is elongate and defines an axial passage 18 (FIGS. 3 and 4) extending between opposite proximal and distal open ends of the tube. The main tube 12 may be graduated with distance indicia (not shown) to facilitate proper intubation. Although the feeding tube assembly illustrated herein is of the type inserted into a human patient through the nostril, it will be understood that a bolus tube assembly could be inserted in other ways (e.g., through the mouth), and/or for purposes other than feeding. Moreover, the patient may be a non-human animal. In one example, the main tube 12 may have a length of between about 36 in and about 55 in and, although it may be of other lengths without departing from the scope of the invention. The main tube 12 may be composed of a thermoplastic polyurethane elastomer, more specifically an aromatic, polyether-based thermoplastic polyurethane, and a radiopaque substance, such as barium, and may be formed by an extrusion process. The main tube 12 may be composed of other material and may be formed in other ways without departing from the scope of the present invention.

Referring to FIGS. 1, 2 and 4, the inlet adaptor 14 includes first and second inlet ports 20, 22, respectively, in fluid communication with a single outlet port 24 (FIG. 4). Each of the inlet ports 20, 22 includes a removable cap(s), 23a, 23b, 23c respectively, secured to the adaptor 14. The caps 23a, 23b are used to plug the inlet ports 20, 22 when they are not being used. The cap 23b has a central opening in it that permits, for example, injection of liquid with a syringe (not shown) into the adaptor 14 via the port 22 through the cap 23b. The cap 23c is used on the cap 23b to plug the central opening in the cap 23b, completely closing off the port 22. In one example, the first inlet port 20 may be used for connection to a source of liquid nutrients, such as a tube extending from a bag of nutrients. The second inlet port 22 may be used for connection to a source of medicine, such as a syringe for injecting medicine. The inlet adaptor 14 may take on other shapes, sizes and configurations (or may be entirely omitted) without departing from the scope of the invention. The inlet adaptor 14 is secured to the proximal end of the main tube 12 at an adaptor weld 26 so that the outlet port 24 of the adaptor is in sealed fluid communication with the axial passage 18 of the main tube. The adaptor weld 26 tapers distally from the adaptor 14 to the main tube 12 so that the weld has a smooth, generally continuously decreasing diameter. It is understood that the adaptor 14 may be secured to the main tube 12 in other ways without departing from the scope of the invention. For example, the inlet adaptor 14 may be secured to the main tube 12 by solvent bonding, or other securement techniques. The adaptor 14 may be composed of the same material as the main tube 12, a blend of materials or a different but compatible material. In one example, the adaptor 14 is composed of blend of polyvinyl chloride and polyurethane elastomer. In another example, the adaptor 14 is composed of an aromatic, polyether-based thermoplastic polyurethane or DEHP-free PVC. The adaptor may be formed from other types of materials within the scope of the invention.

Referring now to FIGS. 1-3, the head component 16 includes a proximal end margin welded to the distal end of the main tube 12 at a main-head weld 28. The main-head weld tapers proximally to the exterior of the main tube 12 so that the weld 28 has a smooth, generally continuously decreasing diameter. This smooth profile provides more comfort to the patient when inserting and removing the feeding tube assembly 10 from the stomach, esophagus and nasal cavity. The head component 16 defines a head lumen 30 in fluid communication with the axial passage 18 of the main tube 12. The head lumen 30 has a diameter that is substantially the same as a diameter of the axial passage 18 of the main tube 12. Generally aligned outlet openings 32 in fluid communication with the head lumen 30 extend radially outward from the head lumen 30 through the head component 16. The outlet openings 32 allow fluid fed into the inlet adaptor 14 to flow through the axial passage 18 of the main tube 12, through the head lumen 30 and out of the outlet openings 32 and into a patient's stomach.

Referring still to FIGS. 1-3, the head component 16 also includes a plurality of cylindrical, generally axially aligned weights 34 (i.e., six weights) received in a weight chamber 36 of the head component 16. In the illustrated embodiment, the weights 34 (broadly, “filler members”) add significant additional weight to the head component 16. In other embodiments (not shown), filler members may be added to the head component that do not significantly increase its weight. In that case, the filler members may serve another purpose, such as rigidifying the head component. A proximal seal 38 is disposed between the head lumen 30 and the weight chamber 36 to prevent fluid from flowing from the head lumen into the weight chamber. The proximal seal 38 has a generally smooth exterior surface and a diameter that is generally about the same as an outer diameter of the weight chamber 36 to provide comfort to the patient when removing and inserting the tube assembly 10.

Referring still to FIGS. 1-3, the head component has a rounded distal tip 40 and a smooth exterior surface. The tip acts as a distal seal, whereby the tip and the proximal seal 38 retain (i.e., trap) the weights 34 within the weight chamber 36 and seal them from the surrounding environment. A maximum diameter of the tip 40 may be slightly larger than the outer diameter of the weight chamber 36. A transition between an exterior surface of the tip 40 and an exterior surface of the weight chamber 36 is smooth and continuous to provide comfort to the patient when removing and inserting the tube. As will be explained in more detail below, the tip 40 may be formed by molding the proximal end portion of the head component 16 using high frequency energy (e.g., radiofrequency energy), although other ways of forming the tip are within the scope of the invention.

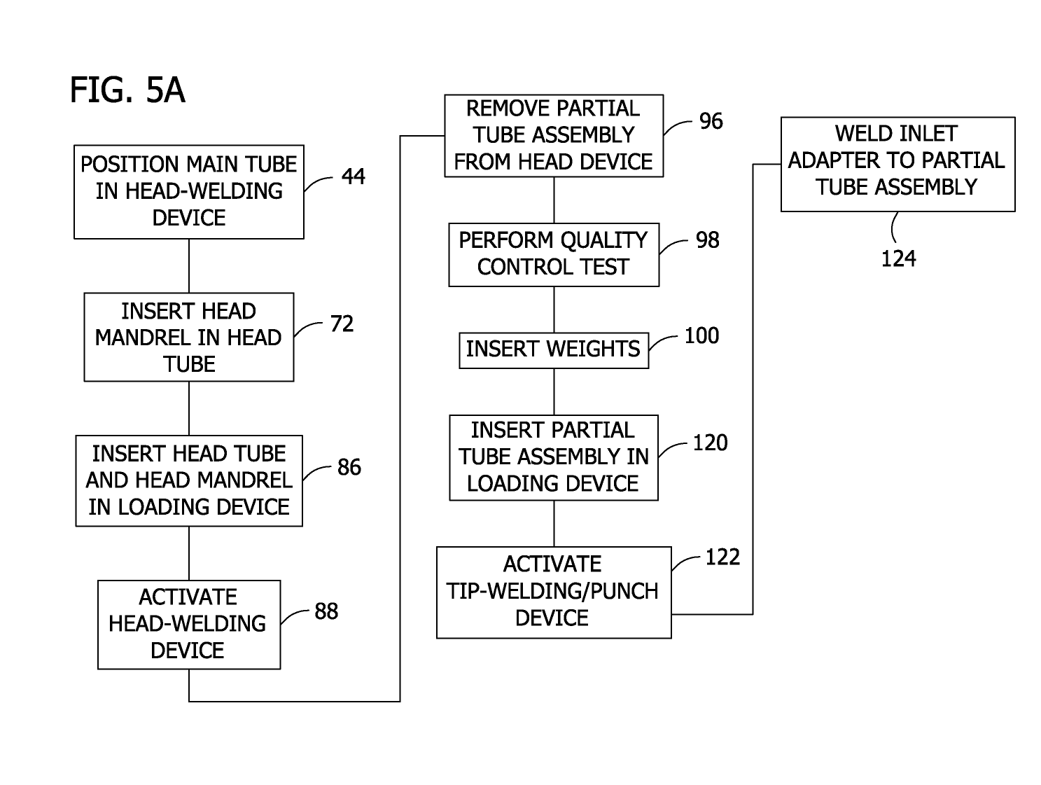

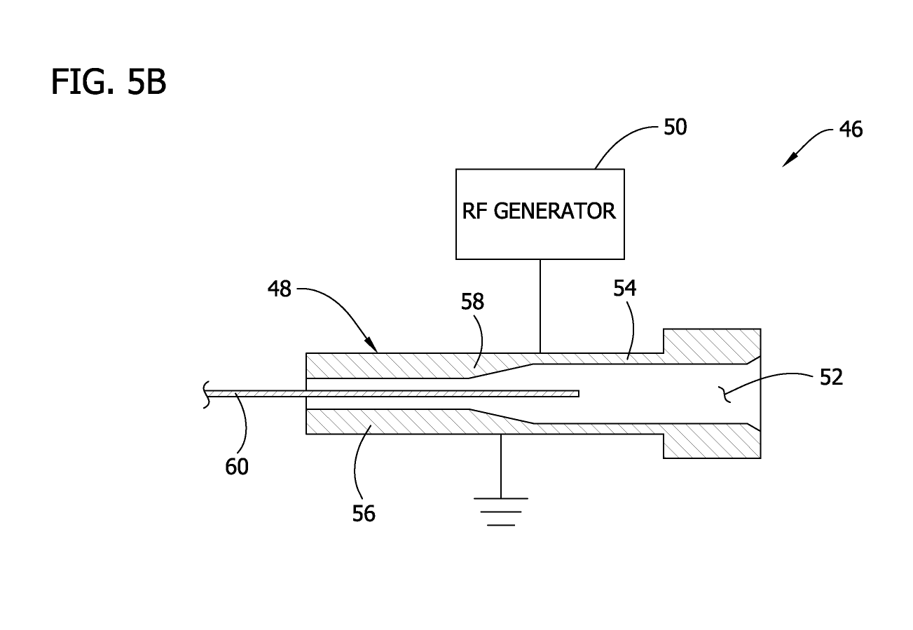

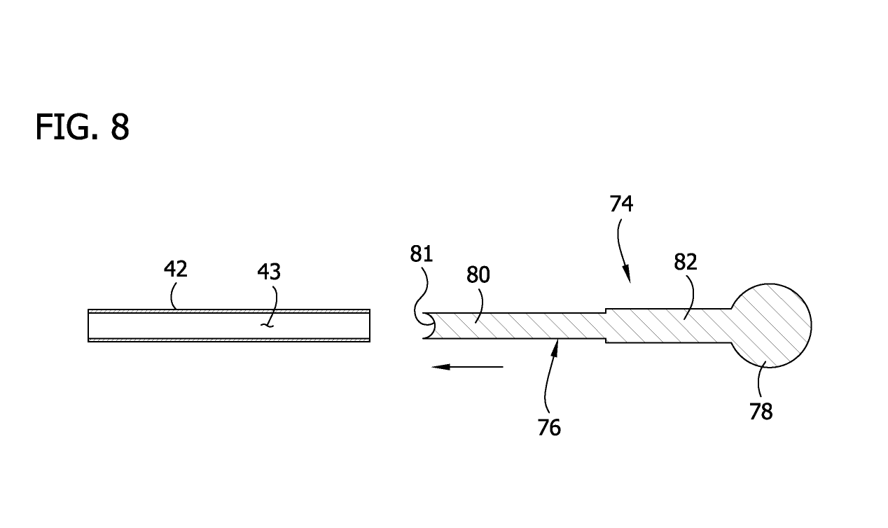

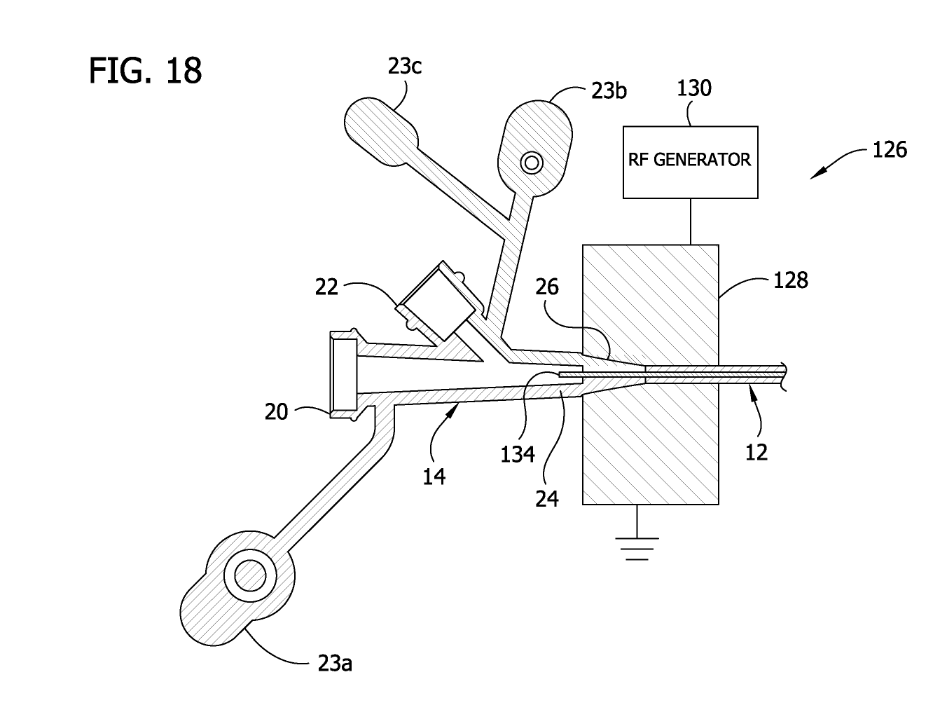

Referring now to FIGS. 5A-19, an exemplary process of forming the nasogastric feeding tube assembly 10 will now be described. In this example, a head tube 42 (FIG. 8) which is used to form the head component 16, and the main tube 12 are preformed. As described above, the main tube 12 may be formed by an extrusion process and may have pre-printed graduated indicia. The head tube 42 has a desired length, such as 2.75 in (6.99 cm) and has axial passage 43 with opposite open ends. In one example, the head tube 42 is composed of the same material or similar material as the main tube 12, such as an aromatic, polyether-based thermoplastic polyurethane. At step 44 (FIG. 5A), the main tube 12 is positioned in a head-welding device, generally indicated at 46 (FIGS. 5B-7 and 9-11). The head-welding device is used to simultaneously weld the head tube 42 to the main tube 12, form the head lumen 30 and form the proximal seal 38 between the head lumen and the weight chamber 36. The head-welding device 46 includes a head die, generally indicated at 48, surrounded by an induction coil (not shown). The induction coil is electrically connected to a radiofrequency (RF) generator 50 (broadly, a source of high frequency energy). In use, radiofrequency current supplied to the induction coil from the RF generator produces a magnetic field that induces eddy currents in the head die 48 which rapidly heat the die. The head die 48 is preferably constructed of steel or other electromagnetic materials, although it may be constructed of other materials within the scope of the invention. As used herein, “high frequency welding” is intended to cover ultrasonic welding in addition to electromagnetic welding. It will be understood that the die can be heated in other ways, such as by electrical resistance heating, without departing from the scope of the present invention. The head die 48 has a cavity 52 having open distal and proximal ends. A first, distal section 54 of the die 48 defines a relatively large diameter part of the cavity 52. A second, proximal section 56 of the die 48 defines a smaller diameter part of the cavity 52. A tapering section 58 of the die 52, extending between the distal and proximal sections 54, 56, respectively, defines a tapering diameter part of the cavity 52. A generally rigid, elongate main-tube mandrel 60 extends from outside of the die 48 through the proximal section 56 so that a distal end margin of the mandrel is disposed within the die. The mandrel 60 may have a constant diameter or may have a different (e.g., smaller) diameter away from the die. The main-tube mandrel 60 is generally concentric with the cavity 52 of the die 48. The mandrel 60 is sized and shaped to be snugly received in the axial passage 18 of the main tube 12.

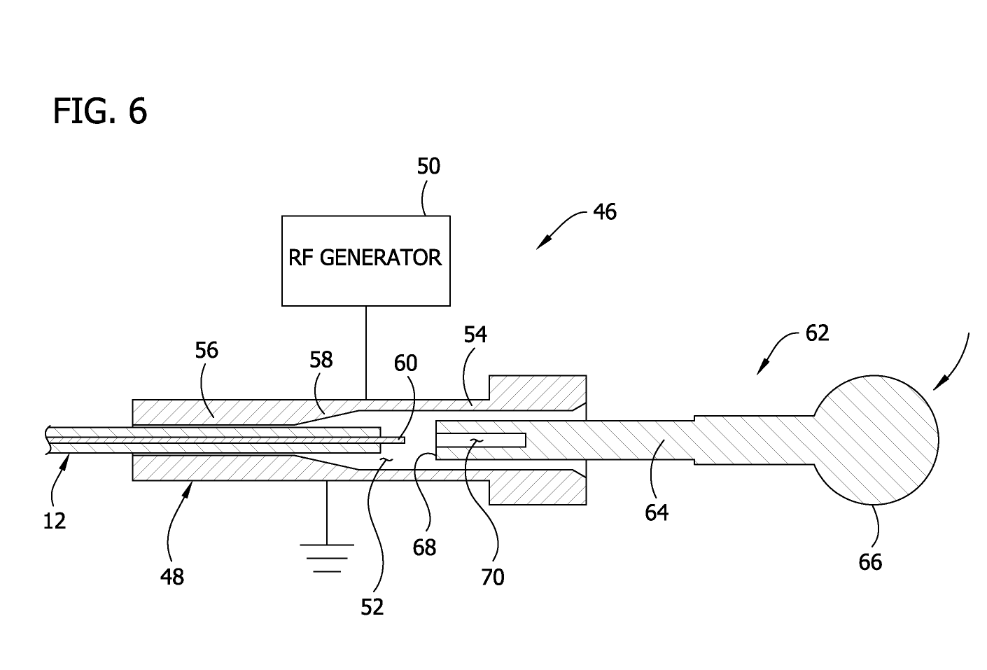

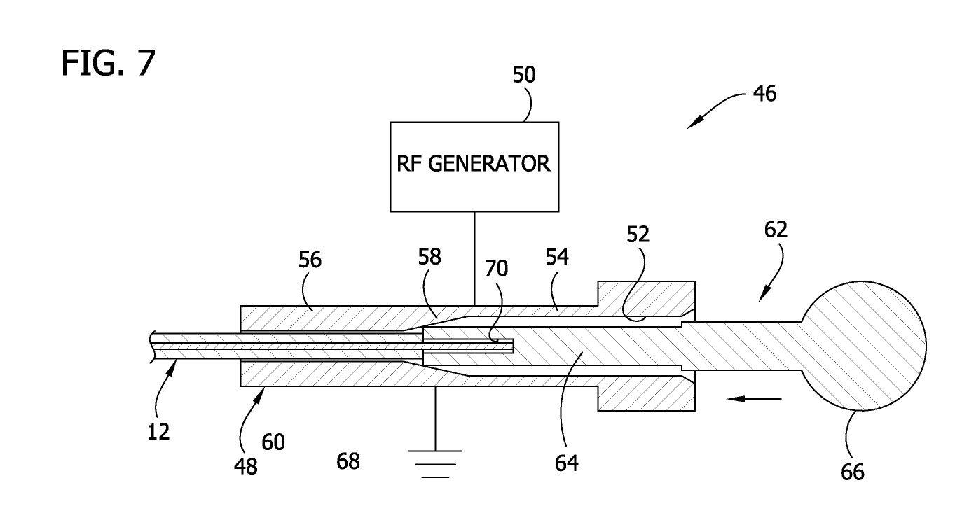

Referring FIGS. 6 and 7, the main tube 12 is inserted into the open distal end of the die 48 and received on the main-tube mandrel 60. The main tube 12 is positioned along the length of the main-tube mandrel 60 by sliding the tube along the length of the mandrel and using a main-tube positioning tool, generally indicated at 62, to properly position the main tube on the mandrel so that the distal end of the mandrel extends out of the distal end of the main tube. In one embodiment, the main-tube mandrel 60 extends a distance between about 0.195 in (0.495 cm) and about 0.205 in (0.521 cm) from the distal end of the main tube 12. The main-tube positioning tool 62 of the illustrated embodiment includes a cylindrical shaft 64 extending from a handle 66. A free end of the shaft 64 defines a contact surface 68 for engaging the distal end of the main tube 12. A mandrel receptacle 70 extends axially through the contact surface 68 and is sized and shaped to receive the distal end margin of the main-tube mandrel 60 but not the main tube 12. A length of the receptacle 70 is selected to be the distance the main-tube mandrel 60 extends from the distal end of the main tube 12 when the main tube is properly positioned on the mandrel.

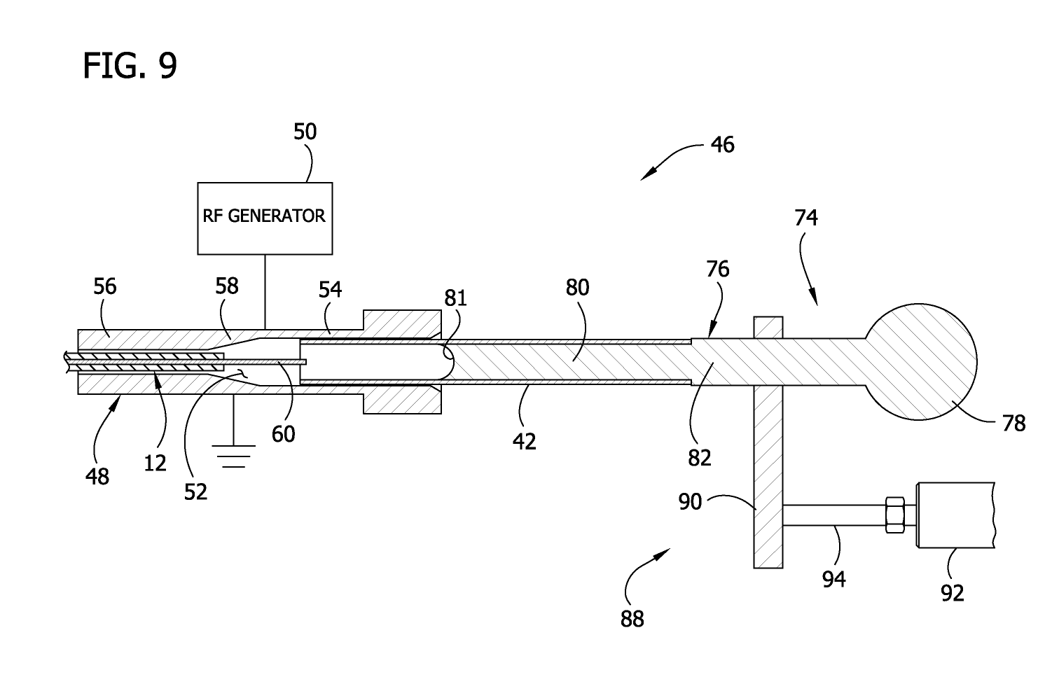

Initially, the main tube 12 is intentionally inserted over the mandrel 60 so that its distal end if closer than desired to the distal end of the mandrel and projects into the larger diameter part of the cavity 52 in the distal section 54 of the head die 48. As shown in FIGS. 6 and 7, to properly position the main tube 12 on the main-tube mandrel 60, the main-tube positioning tool 62 is inserted into the head die 48 through the open distal end. As the positioning tool 62 is being inserted, the contact surface 68 of the tool engages the distal end of the main tube 12 and the main-tube mandrel 60 is received in the receptacle 70 of the tool. As the positioning tool 62 continues to be inserted into the cavity 52, it pushes the main tube 12 back (“proximally”) toward the proximal section 56 of the head die 48. Eventually, the positioning tool 62 bottoms out by engaging the head die in the tapering section 58 (FIG. 7). The main tube 12 is now properly positioned within the head die 48. As shown best in FIG. 9, when the main tube 12 is properly positioned on the main-tube mandrel 60, the distal end of the main tube is disposed generally in the tapering section 58 of the head die 48. After the main tube 12 is properly positioned, the tube is locked in the head die 48, such as by a clamp (not shown), and the main tube positioning tool 62 is removed from the head die. Other ways of locating the main tube 12 in the head die 48 may be used without departing from the scope of the present invention.

At step 72 (FIG. 5A), a head mandrel 74 is inserted in the head tube 42. Referring to FIGS. 8-10, the head mandrel 74 includes a generally cylindrical shaft 76 extending from a bulbous handle 78. The shaft 76 includes a first section 80, including a free end 81 of the shaft, having a diameter that is sized and shaped to be snugly received in the head tube 42. The free end 81 of the shaft 76 is generally concave. The shaft 76 also includes a second section 82 adjacent to the handle 78 having a diameter larger than the first section 80 that is sized and shaped to prevent reception in the head tube 42, thereby acting as a stop. For reasons described below, when the head mandrel 74 is fully received in the head tube 42 (i.e., the second larger section 82 of the mandrel contacts the head tube), the free end 81 of the mandrel does not extend through the head tube and is spaced from an end of the head tube. With the head tube 42 received on the head mandrel 74, a layer of liquid silicone is applied to the exterior proximal end margin of the head tube at step 84 (FIG. 5A) to facilitate withdrawal of the head tube 42 from the head die 48 after welding.

Referring to FIGS. 5A and 9, at step 86, the head tube 42 and the associated head mandrel 74 are received in a head-tube ram (broadly, “a driver”), generally indicated at 88, of the head welding device 46. The head-tube ram 88 includes a head-tube clamping member 90 for securing the head tube 42 and the head mandrel 74 thereto. The head-tube clamping member 90 is moveable and configured to move the head tube 42 secured to the clamping member 90 axially into the head die 48. A cylinder 92, having a linearly moveable piston 94 secured to the clamping member 90, drives the clamping member along a guide rails (not shown). Other ways of moving the head tube 42 into and out of the head die 48 are within the scope of the invention.

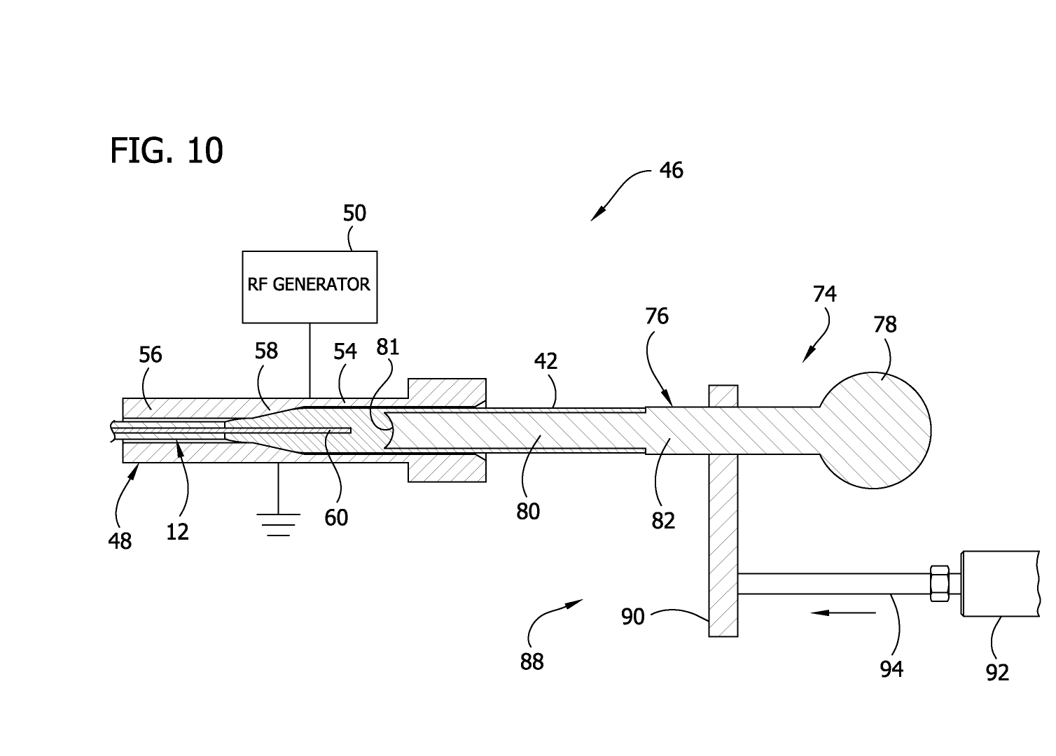

Referring to FIGS. 5A and 10, at step 88, the RF welding device 46 is activated so that the ram 88 moves the head tube 42 into the head die 48 and the die is rapidly heated to a temperature suitable for melting the main tube 12 and the head tube using RF energy from the RF generator 50. As the head tube 42 moves over and receives a portion of the main tube 12, and moves into the tapered diameter of the cavity 52 of the head die 48, the two tubes melt into an intermingled molten mass. The ram 88 of the welding device 46 limits the movement of the head tube 42 into the die 48 so that the distal end of the main-tube mandrel 60 is spaced from the concave end of the head mandrel 74. A portion of the head tube 42 that is disposed between the main tube 12 and the concave free end 81 of the head mandrel 74 melts and flows around the portion of the main-tube mandrel 60 that is extending out of the main tube and flows in a space between the distal end of the main-tube mandrel and the concave free end of the head mandrel. After a suitable elapse of time to allow for the above occurrences to take place, the tubes 12, 42 are cooled inside the head die 48, such as by using pressurized air to cool the die. As the tubes 12, 42 cool and solidify, the head tube welds to the main tube, the head lumen 30 is formed and the proximal seal 38 is formed. Also, the weight chamber 36 is partially defined by the portion of the head tube 42 surrounding the head mandrel 74. The portion of the head tube 42 surrounding the first section 80 of the head mandrel 74 may melt within the die 48 and plastically deform around the mandrel, or this portion may not plastically deform. After completion of this step, a partial tube assembly is formed.

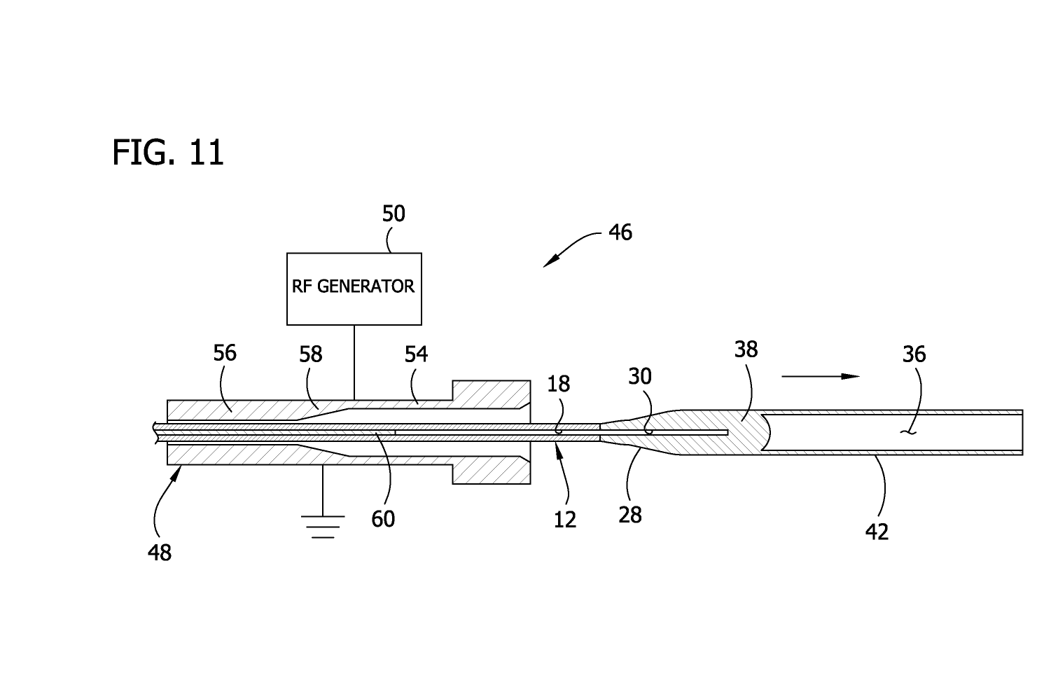

Referring to FIGS. 5A and 11, after cooling in the head die 48, the partial tube assembly is pulled off the main-tube mandrel 60 and removed from the die at step 96. Removal of the main-tube mandrel 60 from the collapsed head tube 42 leaves the head lumen 30 that is in fluid communication with the axial passage 18 of the main tube 12, but is blocked from the weight chamber 36 by the proximal seal 38. At step 98 (FIG. 5A), a quality-control test is performed to ensure that the main-head weld 28 between the main tube 12 and the head tube 42 is air-tight and the partially defined weight chamber 36 is not in fluid communication with the head lumen 30. Pressurized air from a source of pressurized air (not shown) may be introduced into the axial passage 18 of the main tube 12 through the proximal end of the main tube. The partially formed head component, including the entire main-head weld 28, is submerged in water. If the no air bubbles are present in the water, then the partial tube assembly passes the test, and remaining assembly steps may be completed. If air bubbles are present, the partial tube assembly fails, and the partial tube assembly is discarded.

For the partial tube assembly that passes the quality control test, the cylindrical weights 34 are next inserted through the distal open end of the head tube 42 and into the weight chamber 36 at step 100 (FIG. 5A). In one example, a device (not shown) for inserting the weights 34 into the chamber 36 is used.

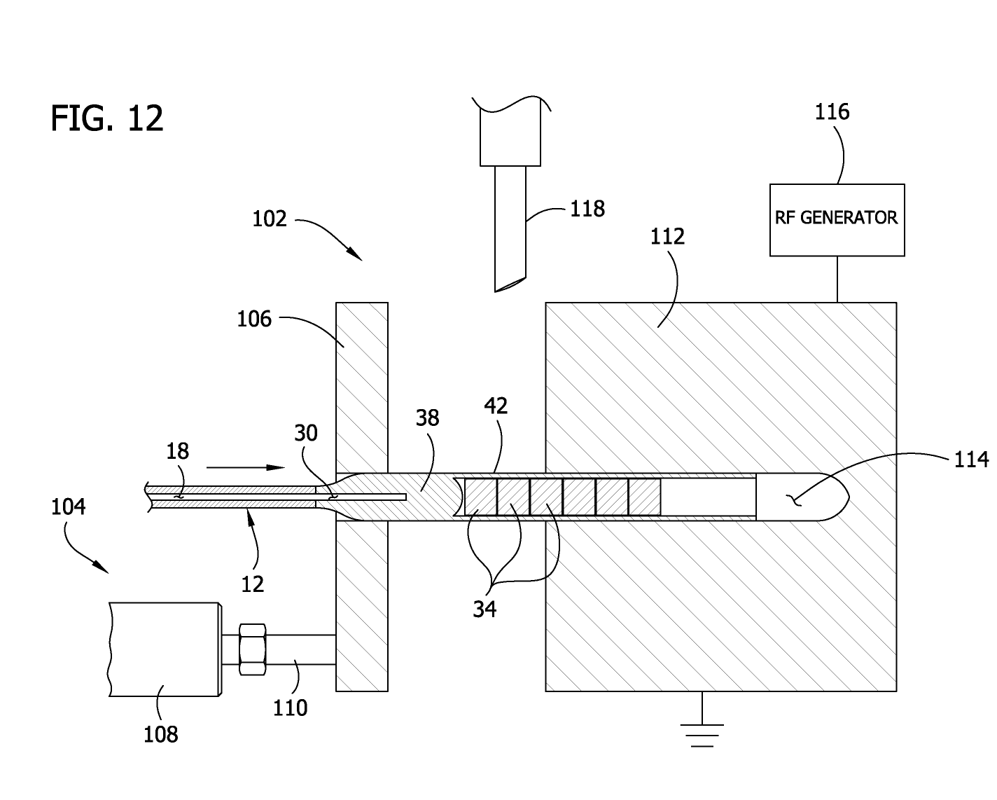

Referring to FIGS. 12-16, after the weights 34 are inserted into the weight chamber 36, the rounded tip 40 and the outlet openings 32 are formed at using a tip-welding/punch device, generally indicated at 102. The tip-welding/punch device includes a ram, generally indicated at 104. The ram 104 is similar to the ram 88 of the head-welding device 46 in that it includes a clamping member 106 for securing the partial tube assembly to the ram and a cylinder 108, having a piston attached to the clamping member 106, for linearly moving clamping member. A tip-die 112 of the tip-welding/punch device 102 defines blind bore 114 having a rounded closed end. The tip-die 112 is heated by radiofrequency induction heating, similar to the head die 48. An induction coil (not shown) surrounding the tip-die 112 is electrically connected to a source of RF energy 116. Radiofrequency current supplied to the induction coil from the source of RF energy 116 produces a magnetic field that induces eddy currents in the tip-die. The eddy currents rapidly heat the tip-die. Other ways of heating the tip-die 128 are within the scope of the invention. The tip-welding/punch device 102 also includes a punch 118 to form the outlet openings 32, as will be further described hereinafter.

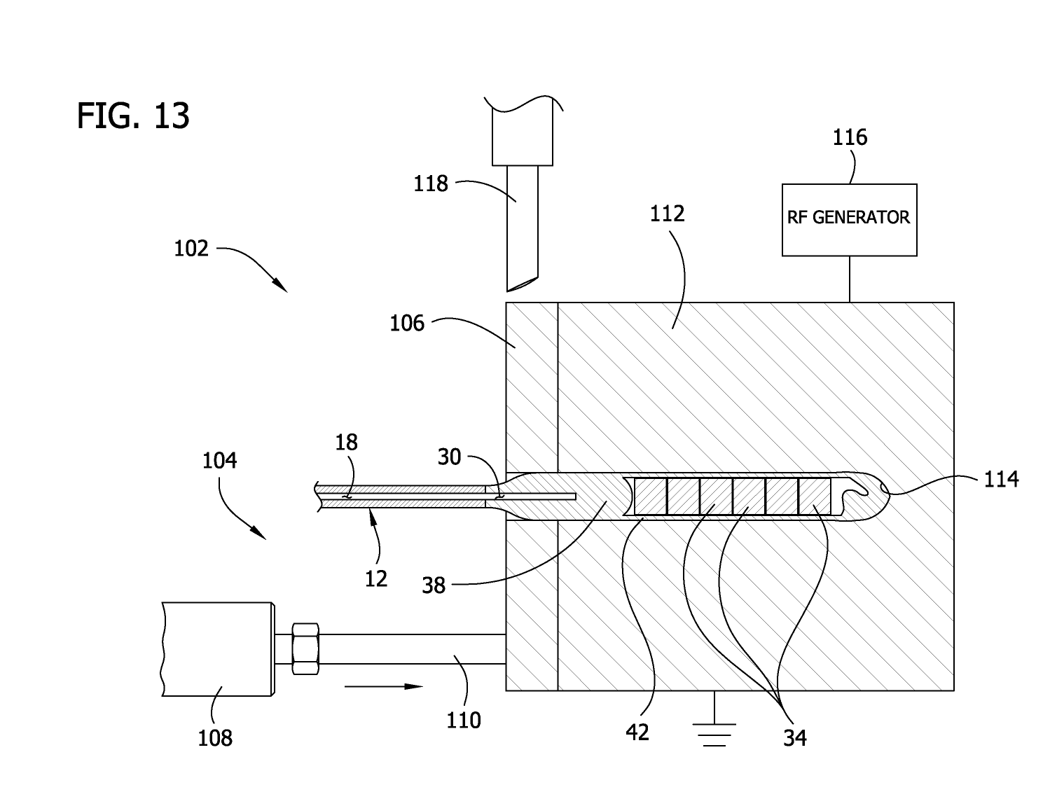

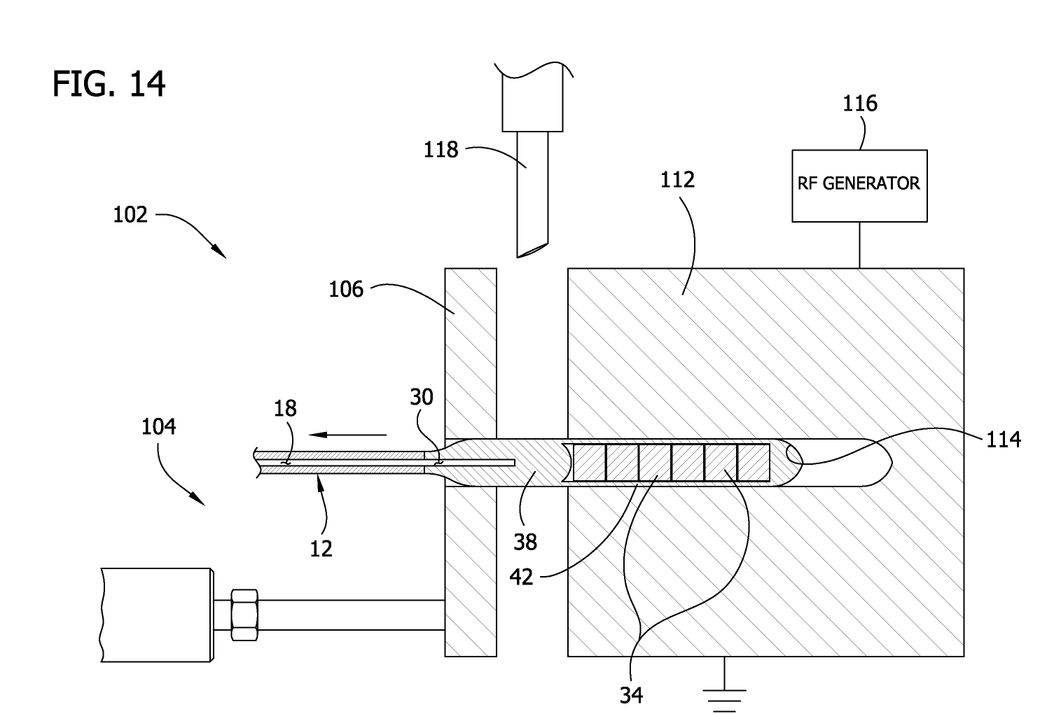

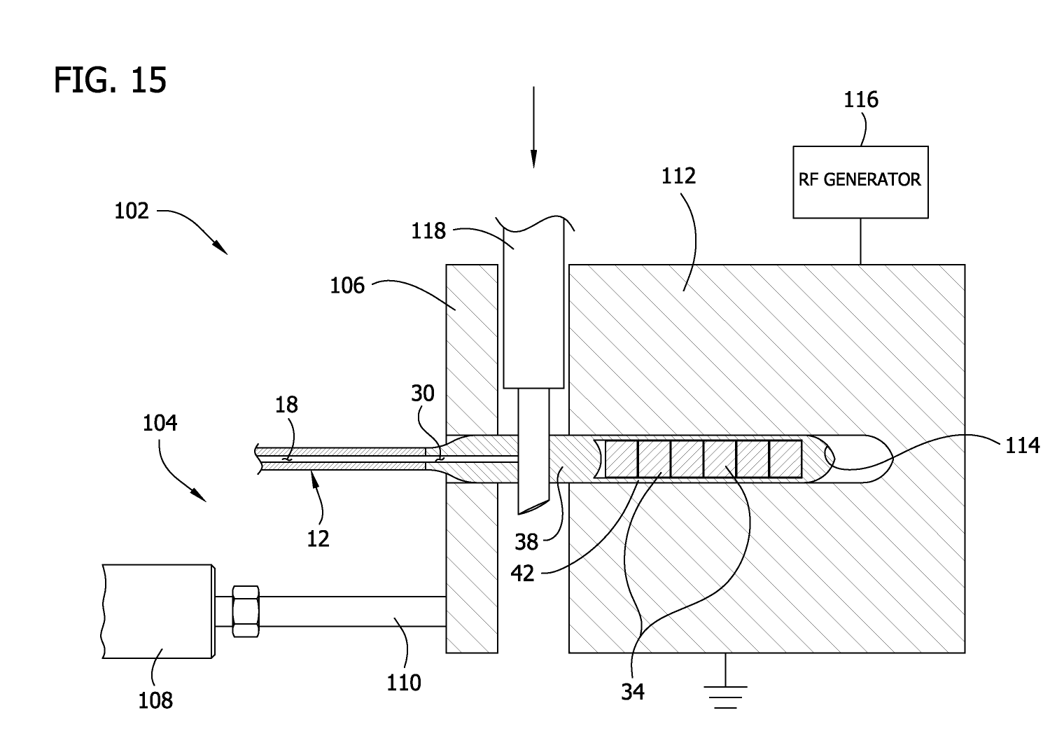

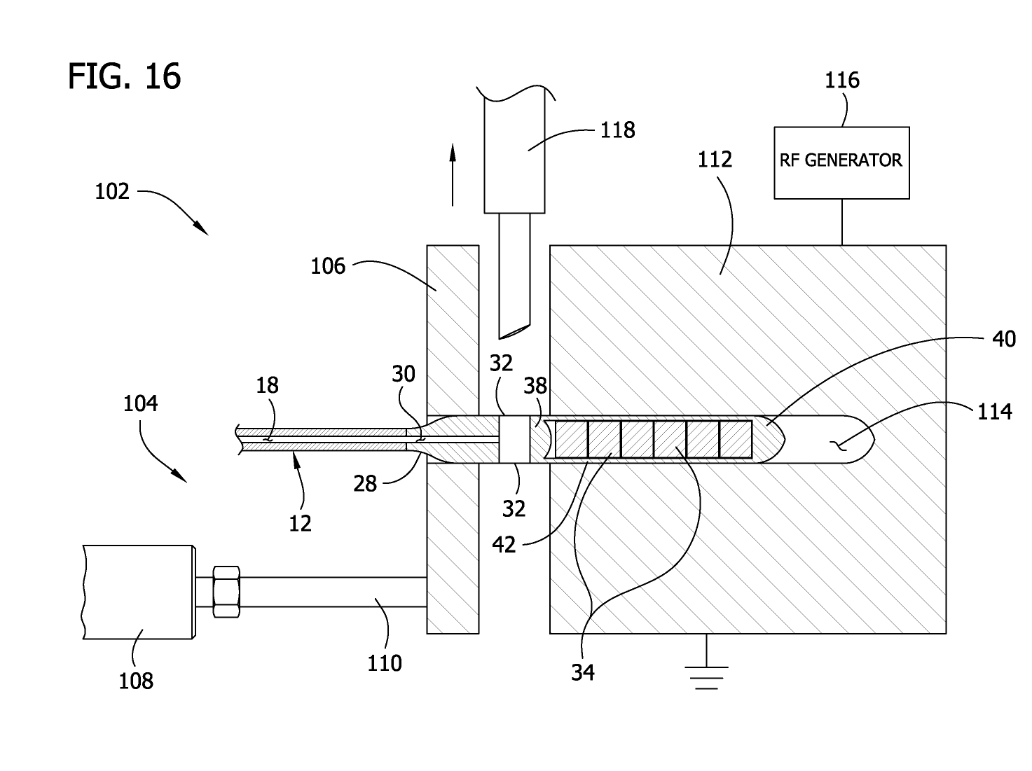

Referring to FIGS. 5A and 12, at step 120 the partial tube assembly, more specifically the head tube 42, is secured in the clamping member 106 of the loading device 102. After the head tube 42 is secured to the clamping member 106, at step 122 the tip-welding/punch device 102 is activated. Referring to FIG. 13, the ram 104 moves the distal end margin of the head tube 42 into the tip-die 112 and the tip die is heated by RF energy from the RF generator 116. As the head tube 42 is forced into the tip-die 112, the distal end portion of the head tube melts is reformed into the rounded shape of the die. The weights 34 in the weight chamber 36 maintain the head tube in its generally cylindrical shape. The head tube 42 is then cooled in the die 112, such as by using pressurized air, to solidify the rounded distal tip 40 of the tube assembly 10. Referring to FIGS. 14 and 15, after the tip 40 is formed, the ram 104 slightly withdraws the partial tube assembly and the punch 118 of the tip-welding/punch device 102 punches through the head tube 42 at or generally adjacent to the head lumen 30 to form the outlet openings 32 in fluid communication with the head lumen. After the punch withdraws (FIG. 16), the partial tube assembly is removed from the tip-welding/punch device 102. It is understood that the tip welding and punch steps may be performed using a separate device or at different times in the manufacturing process without departing from the scope of the present invention. The assembled head component 16 has a length of between about 1.5 in (3.81 cm) and about 2.0 in (5.08 cm). The head component 16 can be coated with a hydromer or other lubricating material that facilitates sliding the head component into the body of the patient.

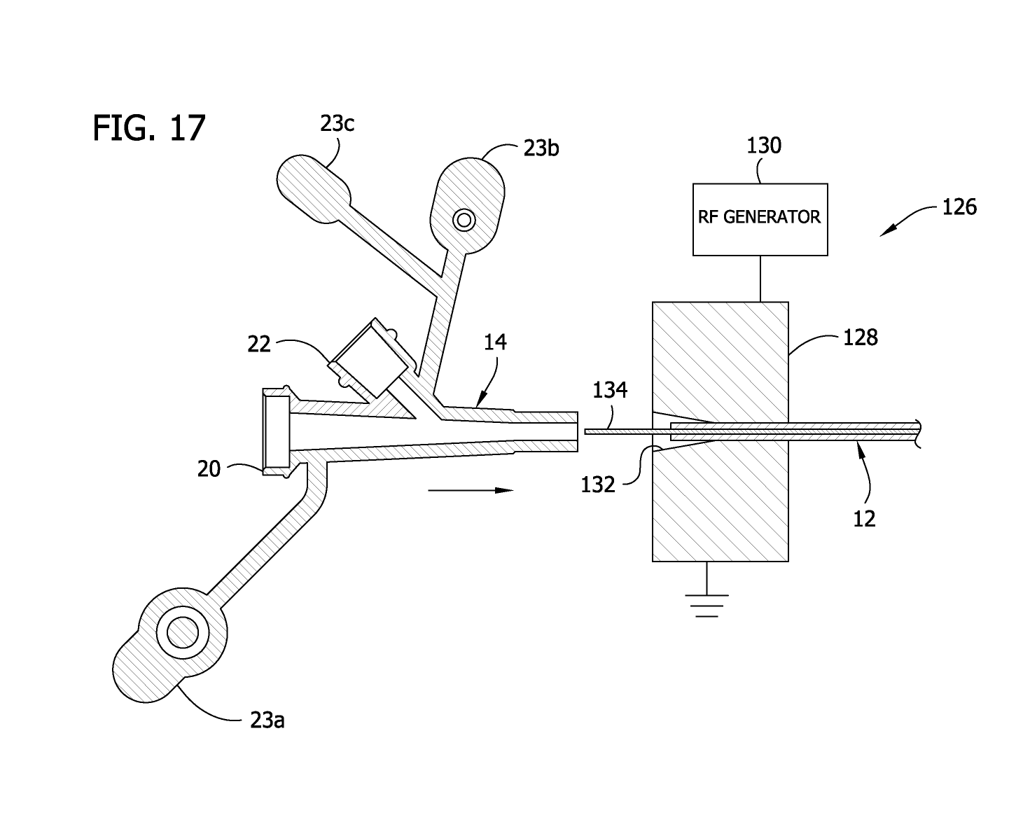

Referring to FIGS. 5A, 17 and 18, at step 124 the inlet adaptor 14 is welded to the proximal end of the main tube 12. The adaptor 14 is welded the main tube 12 using an adaptor welding device 126. The construction and operation of the adaptor welding device 126 is similar to that of the head welding device 46. The adaptor welding device 126 includes an adaptor die 128 that is heated by radiofrequency induction heating, similar to the head die 48 of the head-welding device 46. That is, an induction coil (not shown) surrounds the adaptor die 128 and is connected to a source of RF energy 130. Radiofrequency current supplied to the induction coil from the source of RF energy 130 produces a magnetic field that induces eddy currents in the die 128, which rapidly heat the die. Other ways of heating the adaptor die 128 are within the scope of the invention. The die 128 defines a tapered cavity 132 (FIG. 17). The main tube 12 is received on a mandrel 134 of the device so that the proximal end of the main tube projects into the tapered cavity 132 of the die 128. The adaptor 14 is then inserted into the adaptor die 128, such as by an adaptor ram (not shown) similar to the rams of the head welding device 46 and the tip-welding/punch device 102. The adaptor welding device 126 is activated to heat the adaptor die 128 to a suitable temperature. The adaptor 14 is moved into the die 126 and over the proximal end margin of the main tube 12 in the tapered cavity 132 of the die, and the inlet adaptor and the main tube melt together. After a suitable elapse of time, the inlet adaptor 14 and the main tube 12 are cooled in the die, forming the adaptor weld 26. It is understood that the inlet adaptor 14 may be secured to the main tube 12 in other ways besides RF welding without departing from the scope of the present invention. For example, the inlet adaptor 14 may be secured to the main tube 12 by solvent bonding or other bonding techniques.

Having described embodiment(s) of the invention in detail, it will be apparent that modifications and variations are possible without departing from the scope of the invention defined in the appended claims.

When introducing elements of the present invention or the embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions, products, and methods without departing from the scope of the invention, it is intended that all matter contained in the above description and shown in the accompanying drawings shall be interpreted as illustrative and not in a limiting sense.

What is claimed is:

1. A method of forming a tube assembly with a bolus head component comprising:
providing a head tube with an axial passage having an open proximal end and an open distal end;
welding the proximal end margin of the head tube to a distal end margin of a main tube including collapsing a proximal portion of the head tube axial passage;
forming a head lumen in the collapsed proximal portion of the head tube, the head lumen being in fluid communication with an axial passage of the main tube and being sealed from fluid communication with a distal portion of the axial passage in the head tube;
sealing a distal end of the head lumen to prevent fluid communication between the head lumen and a distal portion of the axial passage of the head tube disposed distally of the seal;
inserting at least one filler member into the distal portion of the axial passage of the head tube through the distal open end of the head tube; and
closing the distal open end of the head tube.

2. A method as set forth in claim 1 wherein welding the proximal end margin, forming a head lumen, and sealing the head lumen are performed substantially simultaneously.

3. A method as set forth in claim 2 wherein welding the proximal end margin, forming a head lumen, and sealing the head lumen comprise heating the head tube and pressing the main tube and the head tube together in a die.

4. A method as set forth in claim 3 wherein heating the head tube is performed using high frequency energy.

5. A method as set forth in claim 3 wherein the die is heated using radiofrequency energy.

6. A method as set forth in claim 5 further comprising positioning the main tube on the mandrel so that a selected length of the mandrel projects from the main tube.

7. A method as set forth in claim 3 wherein forming a head lumen further comprises melting the head tube around a mandrel extending outward from the distal end of the main tube.

8. A method as set forth in claim 7 wherein sealing the head lumen further comprises melting the head tube between a distal end of the main tube mandrel and a proximal end of a head tube mandrel spaced from the distal end of the main tube mandrel.

9. A method as set forth in claim 1 further comprising forming at least one radial opening through the head tube in fluid communication with the head lumen.

10. A method as set forth in claim 9 wherein forming at least one radial opening comprises punching through the head tube at the head lumen to form opposite openings extending generally radially from the head lumen.

11. A method as set forth in claim 1 wherein closing the distal end of the head tube comprises heating the distal end margin of the head tube and molding the distal end margin into a rounded tip.

12. A bolus tube assembly comprising:
an elongate main tube having an axial passage extending to an open distal end of the main tube;
a bolus head component welded to the distal end margin of the main tube by high frequency energy at a main-head weld, the head component being formed from a head tube, the head component including a head lumen in fluid communication with the axial passage of the main tube, and a chamber, the head lumen being formed within the head tube by material of the head tube melted to seal the head lumen from a portion of the tube defining the chamber, a distal end of the head tube being deformed to close off the distal end and seal the chamber between the head lumen and the distal end;
at least one filler member enclosed within the chamber.

13. A bolus tube assembly as set forth in claim 12 further comprising a transverse opening in the head tube in communication with the head lumen and opening radially of the head component.

14. A bolus tube assembly as set forth in claim 13 wherein the transverse opening extends through an entire width of the head component.

15. A bolus tube assembly as set forth in 12 further comprising plural filler members in the chamber.

16. A bolus tube assembly as set forth in claim 12 wherein the main-head weld tapers toward the main tube.

17. A bolus tube assembly as set forth in claim 12 further comprising an adaptor welded by high frequency energy to the proximal end margin of the main tube.

18. A bolus tube assembly as set forth in claim 17 wherein a distal end margin of the adaptor tapers toward the main tube.
